(19) United States
(12) Patent Application Publication
(10) Pub. No.: US 2011/0201991 A1
(43) Pub. Date: Aug. 18, 2011

(54) VALVE BYPASS GRAFT DEVICE, TOOLS, AND METHOD

(75) Inventor: James L. Pokorney, Northfield, MN (US)

(73) Assignee: CARDIOUS, INC., St. Paul, MN (US)

(21) Appl. No.: 13/094,445

(22) Filed: Apr. 26, 2011

Related U.S. Application Data
(63) Continuation of application No. 10/975,941, filed on Oct. 26, 2004.
(60) Provisional application No. 60/515,833, filed on Oct. 30, 2003.

(51) Int. Cl. A61M 5/00; A61B 17/04; A61B 17/064; A61B 17/08

(52) U.S. Cl. 604/9; 606/228; 606/219; 606/151

(57) ABSTRACT

A medical implant comprises a hollow conduit having a first end opening, a second end opening, and a slit opening located between said first and second end openings, and a one way valve located within said conduit.

### CROSS-REFERENCE TO RELATED APPLICATIONS

This application is a continuation of prior U.S. patent application Ser. No. 10/975,941 filed Oct. 26, 2004 and entitled “Valve Bypass Graft Device, Tools, and Method,” which claims the benefit of U.S. Provisional Patent Application No. 60/515,833 filed Oct. 30, 2003, the entireties of which are incorporated herein by reference.

### FIELD OF THE INVENTION

This invention relates to an improved implant, improved implant tools, and an improved implant technique for the interposition of an extracardiac conduit between the left ventricle of a beating heart and the aorta to form an alternative one-way blood pathway thereby bypassing the native diseased aortic valve.

### BACKGROUND OF THE INVENTION

A reduction in the heart's cardiac output, that is, the reduced ability of the heart to output oxygenated blood from the left side of the heart can result from various abnormalities and diseases. In most cases, this reduction in output is due to aortic valve disease. The major type of aortic heart valve disease is valve stenosis. Stenosis involves the narrowing of the aortic outflow tract. The stenosis typically involves the buildup of calcified material on the valve leaflets, causing them to thicken, impairing their ability to fully open to permit adequate forward blood flow.

Stenosis of the aortic valve obstructs flow leaving the ventricle. This obstruction of the outflow tract can ultimately lead to hypertrophy of the left ventricle, meaning the size of the ventricular chamber becomes enlarged. This condition leads to diastolic dysfunction of the left ventricle, that is, an impaired ability of the left ventricle to adequately fill with blood. Diastolic dysfunction accounts for about 20% to 40% of heart failures.

Open heart surgical treatment is available to relieve left ventricular outflow tract obstruction due to stenosis. In most cases, the native aortic valve is surgically removed and replaced with a prosthetic or man-made valve. Valve replacement surgery has been performed for over 40 years and is considered the most effective therapy for outflow tract obstruction even though the technique is far from perfect. A particular drawback of the conventional aortic valve replacement procedure is that it requires the patient to be placed on a heart-lung machine wherein the heart and lungs are stopped. Open-heart surgery on a still heart involves the use of cardiopulmonary bypass, aortic cross-clamping and cardioplegic arrest. The risks and complications associated with this highly invasive procedure are well known. The most serious risks of cardiopulmonary bypass and aortic cross-clamping are the increase in the likelihood of bleeding and stroke. A stroke is an occlusion of an artery in the brain. It can be caused by particles or emboli generated during the heart valve procedure. Emboli can be generated as calcific particles due to the necessary manipulation of a calcified aorta or valve, or emboli can be generated in the form of blood clots caused by the blood's interaction with the foreign surfaces of the heart-lung machine. Also, patients who undergo surgeries using cardiopulmonary bypass often require extended hospital stays and experience lengthy recoveries. Therefore, while conventional heart surgeries produce beneficial results for many patients, some patients sustain debilitating injuries or death due to the procedure. Also, numerous other people who might benefit from such surgery are unable or unwilling to undergo the trauma and risks of a conventional stopped heart procedure.

### BACKGROUND-PRIOR ART

Tools and techniques for the interposition of an extracardiac conduit between the left ventricle and the aorta have been evolving over the last century. The invention presented herein is another step in that evolution. Compared to prior art, this invention advances the state of the art by creating an innovative implant, implantation tools, and implantation method that eliminate the major reasons why this procedure has not been widely used to date. In particular, the invention, compared to prior art, substantially reduces the potential for excessive blood loss or the generation of stroke causing emboli. Also, the invention reduces the possibility of inflicting damage to the heart, maximizes blood flow through the implant, and protects the implant from kinking or crushing blows.

The following is a chronology of major developments in this field of art.

In 1910, Alex Carrel first described the idea of creating a bypass from the left ventricular cavity to the descending aorta by employing paraffin rubber tubes and jugular vein (Carrel, A.: Experimental Surgery of the Aorta and Heart. Ann. Surg., 52:83-95, 1910).

In 1923, Jeger improved upon these experiments by inserting a valve bearing conduit between the ventricle and aorta and managed to keep an animal alive for four days using such a device, even though the ascending aorta was completely ligated (cited in Kuttner, H.; Chirurgische Operationslehre. Ed. 5. Leipzig, Barth, Vol. 2, 1923).

By 1955, Sarnoff and his co-workers were able to report on seven long term dog survivors with valve bearing conduits inserted between the apex of the left ventricle and the aorta (Sarnoff, S. J., et al.: The Surgical Relief of Aortic Stenosis by Means of Apical-Aortic Valvular Anastomosis. Circulation, 11:564-5575, 1955).

The technique was first applied clinically in humans in the early 1960's by Templeton, who inserted a completely rigid conduit/valve device into five patients with aortic stenosis, interposing this prosthesis between the apex of the left ventricle and the thoracic aorta One of these patients is said to have survived for a 13 year period (cited in Dembitsky WP, et al.: Clinical experience with the use of a valve-bearing conduit to construct a second left ventricle outflow in cases of unresectable intraventricular obstruction. Ann Surg 184:317, 1976)). It should be noted that these first human procedures, like many heart procedures then, were performed without the benefit of cardiopulmonary bypass (Reder RF, et al.: Left ventricle to aorta valved conduit for relief of diffuse left ventricular outflow tract obstruction. Am J Surg 135:547-542, 1978).

Even though this first human use proved feasibility of the valved conduit device, the procedure did not gain popularity with surgeons because, precisely at that point in the evolution of surgical techniques, cardiopulmonary bypass methods were being perfected which led surgeons to select direct valve resection and replacement over Templeton's more radical valve bypass procedure. The open heart, on pump surgical technique, that is, a technique which requires a stopped heart and external cardiopulmonary support, is still considered the “gold standard” method of choice for the relief of aortic valve stenosis more than 40 years later. Its known problems, namely excessive blood loss and potential stroke due to the need for cardiopulmonary support, have now been widely accepted because no better alternative has emerged for heart valve surgery.

Even though direct valve replacement rapidly became the standard, throughout the remainder of the twentieth century the valved conduit procedure continued to be used in special, limited circumstances.

In 1973, Bowman published on a flexible conduit consisting of a porcine aortic valve sewn into a polyester graft (Bowman F O Jr., et al.: A valve-containing Dacron Prosthesis: its use in restoring pulmonary artery-right ventricular continuity. Arch Surg 107:724, 1973).

Dembitsky reported in 1976 on a flexible valved-conduit graft with the added feature of a rigid ventricular connector covered with a polyester cloth (Dembitsky WP, et al.: Clinical experience with the use of a valve-bearing conduit to construct a second left ventricle outflow in cases of unresectable intraventricular obstruction. Ann Surg 184:317, 1976).

Cooley followed up on Dembitsky's work in 1976 by replacing the cloth covered connector with a pyrolytic carbon connector. Also, the Cooley design added a Teflon felt sewing cuff designed to be sewn to the ventricle wall (Cooley DA, et al.: Surgical treatment of left ventricular outflow tract obstruction with apico-aortic valved conduit. Surgery 80:674, 1976).

Pierce improved on Cooley's cuff design in 1978 by adding felt washers which could be added distal to the felt cuff to adjust the depth of the connector insertion into the ventricle (Pierce W S, et al.: A new prosthesis for reconstruction of the left ventricular outflow tract. Ann Yhor Surg 25:358-363, 1978). Also, Pierce's design, like Dembitsky's, employed a cloth covered rigid stainless steel connector, but he also added a series of perforated holes in the metal to facilitate tissue in-growth into the cloth fabric. Until Pierce, all techniques used a circular cutter, commonly called a cork borer, to remove myocardium tissue at the apex of the ventricle to insert the conduit's ventricular connector. Pierce developed a method to pierce and dilate the myocardium without removing any myocardial tissue. In his technique, a scalpel is used to pierce the ventricular wall and is then removed, then a blunt tipped dilator is inserted and removed before the conduit is finally inserted and sutured in place. This procedure needed to be done on a still, non-beating heart.

In 1978, Murray describes another variation in the procedure where a bioprosthetic valve is sewn directly to the ventricle's epicardial surface directly over a cored opening. The conduit is then sewn from the valve to the aorta Again, this procedure was done “on-pump” (Murray G F: Valve Placement in the Ventricular Apex for Complicated Left Ventricular Outflow Obstruction, Ann. Thor. Surg., Vol 25, No 4, 1978).

Brown, in 1978, developed a method to core out the ventricular tissue while protecting the underlying structures from damage. Brown inserted a Foley catheter into the ventricle through a small stab wound in the apex. The balloon was inflated to form a protective backstop. Traction was placed on the Foley catheter as the cork borer, threaded over the catheter, was advanced through the myocardium to excise a circular piece of myocardium. The balloon, inflated to a diameter larger than the borer, prevented the sharp edge of the borer from damaging papillary muscles and other such structures in the ventricle. His balloon catheter approach would be the basis for developing “off pump” methods a few years later, that is, methods that do not require the use of cardiopulmonary support (Brown J W, et al.: Technique for insertion of apioaortic conduit, Amer. Journal Surg., Vol 76, No. 1, July, 1978).

Norwood, in 1983, describes a procedure specifically planned to be done without cardiopulmonary support. He uses the similar “Foley catheter backstop” method Brown perfected five years earlier except, after removing the cork borer and tissue, the balloon remains in close contact against the ventricular wall while the conduit is threaded over the catheter into the ventricular wall. Only after the conduit is sewn in place is the balloon collapsed and removed. A clamp is used to occlude the conduit once the catheter is removed. This method allowed conduit connection to the ventricle under full blood pressure. One limitation of this approach is that the conduit needs to be in two pieces so that the catheter can be removed. Once removed, the conduits need to be sewn together (Norwood W I, et. al.: Management of infants with left ventricular outflow obstruction by conduit interposition between the ventricular apex and thoracic aorta. J Thorac Cardiovasc Surg 86:771-776, 1983).

Brown, in 1984, performed a series of human procedures without cardiopulmonary support using the “Foley catheter backstop” approach as well, but did not thread the catheter through the conduit. In his version of the procedure, the conduit cuff is loosely sewn into the ventricle wall with substantial slack in the sutures. After the core cut is made using the balloon as a sealing mechanism, the inflated balloon catheter, the cut tissue, and the cork borer are ail removed and the Conduit connector is quickly inserted to fill the gaping hole. The loose sutures are then tightened to complete the connection (Brown J W, et al. Apioaortic valved conduits for complex left ventricular outflow obstruction: technical considerations and current status. Ann Thorac Surg 1984; 38:162-8).

In 2002, a paper was published by Khanna that again demonstrated the “Foley catheter backstop” technique as taught by Norwood. Khanna reported that one drawback to performing the procedure was the possibility that the Foley catheter may be cut, causing immediate deflation and rapid blood loss. Although the surgeon's finger could be used to breach the hole or gap, excessive blood loss using this technique seemed possible if the balloon seal was not maintained (Khanna SK, et al.: Apico-aortic conduits in children with severe left ventricular outflow tract obstruction, Ann Thorac Surg, 2002; 73:81-7).

This possibility was realized the following year. In 2003, Vassiliades reported on the surgical results of three patients using the same balloon catheter sealant technique. There were no hospital deaths, but the average blood loss was 850 cc, demonstrating substantial blood loss is possible using the “Foley balloon backstop” technique ((Vassiliades T A, Off-pump apicoaortic conduit insertion for high-risk patients with aortic stenosis, Euro. J Cardio-Thorac Surg 23 (2003) 156-158).

Looking back on these early pioneering efforts, acceptance of this innovative technique in the 1960's and 1970's was most likely limited because the procedure required some form of cardiopulmonary bypass to be successful. Therefore, it didn't seem logical to surgeons to use this non-anatomic approach to alleviate aortic stenosis when direct valve replacement, considered a more anatomically correct solution, was showing good results. By 1983, Norwood and Brown were showing some success with off pump valved conduit procedures, but the balloon sealant method to control bleeding from the ventricle was not foolproof as demonstrated by Vassiliades in his 2003 paper. Based on reading of prior art, the inability to perform the procedure completely off-pump without any risk of major blood loss may have been the major limiting factor preventing wide acceptance of this procedure. If an off pump procedure, one not requiring any cardiopulmonary support, could be improved to ensure the surgeon that there is little chance of excessive blood loss when connecting the conduit between a pressurized heart and aorta, the procedure could gain more acceptance.

In 2004, Haverich filed a patent application on a valved-conduit implantation method (U.S. patent application 20040162608). His application states many general ideas that are already described in the prior art referenced herein. The broad ideas and proposed concepts outlined in the Haverich application on how to build a valved-conduit implant and how to insert the implant seem too vague to be considered patentable when compared to the specific prior art described over the last 94 years.

In all the prior art reviewed, there were no specific improvements made in the method used to create the aortic connection. In all procedures to date, the aorta is occluded to temporarily isolate the graft from blood flow. In Haverich, a balloon occlusion method is proposed. In all other prior art reviewed, either the aorta is completely clamped upstream from the connection site to stop all flow or a curved side clamp is used to isolate a portion of the aorta being cut from the flow stream. In either case, the aorta is substantially handled and manipulated and then ultimately pinched or clamped, either to fully or partially occlude flow. This clamping action is known to break loose calcified particles from the aortic wall. It is known to all those practicing in the art that these loose particles can migrate to the brain and cause a stroke. Therefore, when compared to “on pump” open heart valve surgery where the aorta is also clamped, the clamping requirements necessary in all prior art procedures most likely did not motivate surgeons to consider switching to this procedure. Conversely, if the procedure could be done without clamping the aorta, the potential for emboli generation would be greatly reduced compared to traditional surgery and the procedure could gain wider acceptance.

Also, in all prior art where the procedure was performed “off pump”, the ventricular connection was made by removing a plug or cored section of myocardium muscle at or near the apex. It would be best to perform the procedure “off pump” without removing any heart tissue to avoid any possible injury to the papillary muscle attachments located nearby within the ventricle.

Quite interestingly, even though this alternative form of valve replacement never gained popularity due to its blood management issues and aortic clamping issues, the clinical results reported by the early pioneers using valved-conduits were very promising. The long term hemodynamic improvement gained by this procedure in all reported studies compared favorably to traditional valve replacement results.

The invention described in this application provides for an improved implant, improved implant tools, and an improved procedure when compared to prior art. The invention minimizes the potential damage to the ventricle, minimizes blood loss during the beating heart procedure, improves the aortic connection method, minimizes potential damage to the aorta and the associated potential of generating emboli, improves blood flow through the implant, and allows the surgeon to perform the procedure quicker, easier, and more predictably.

### BRIEF SUMMARY OF THE INVENTION

The primary object of the present invention is to provide an implantable device, the associated implant tools, and a reliable and safe method to alleviate the problems associated with a stenotic native aortic valve without replacing the diseased valve, without stopping the heart or lungs, without removing myocardial tissue, without excessive blood loss, and without disrupting flow or generating emboli in the aorta.

Specifically, the invention has the following advantages:

The invention minimizes damage to the heart:
by allowing the surgeon to gain access to the ventricle of a beating heart without removing any myocardial tissue to avoid any possible injury to the papillary muscle attachments located nearby within the ventricle.
by preventing the surgeon from inadvertently cutting any nearby ventricular endocardial surfaces or structures when creating a connection to the left ventricle of a beating heart.

It allows the surgeon to connect the implant to a fully pressurized aorta:
without squeezing or clamping the aorta.
without stopping or reducing blood flow through the aorta.

It minimizes blood loss when connecting the implant to either the aorta or ventricle wall
by allowing the surgeon to sew the implant to the aorta before the aorta is cut open.
by providing the surgeon with implant tools that are temporarily positioned within the implant such that blood cannot flow past the tools and out the implant before the surgeon completes the procedure.
by allowing the surgeon to insert or remove the implant tools into or out of the implant without interfering with the one-way valve located within the implant.
by allowing the surgeon to rapidly complete the implant procedure immediately after the implant tools are removed.

It maximizes blood flow through the implant
by ensuring that the surgeon cuts a hole in the aorta that aligns with the internal diameter of the implant opening.
by allowing the surgeon to use a valve that is larger in diameter than the conduits connected to it.
by preventing the flexible conduit from kinking if it is formed in a tight radius or if an external crushing force assaults it.

These and other objects and advantages of this invention are achieved by a prosthetic aortic valve integrated within a flexible conduit which is connected between the ventricle and the aorta such that the stenotic aortic valve is bypassed.

The above mentioned objects and advantages of this invention will become apparent from the following description taken in connection with the accompanying drawings, wherein is set forth by way of illustration and example, preferred embodiments of this invention.

### BRIEF DESCRIPTION OF THE DRAWINGS

In the drawings, closely related figures have the same number but different alphabetic prefixes.

FIG. 1 shows a side view of an implant with two open slits and the associated implant tools prepared for insertion into the implant.

FIG. 2 shows a mixed side view/cross sectional view of an implant connected between the ventricle and the aorta.

FIG. 3A shows a side view and selected end views of an implant.

FIG. 3B is a cross-section of the ventricular end of the implant shown in FIG. 3A.

FIG. 3C is a cross-section view of the valve segment of the implant shown in FIG. 3A.

FIG. 4A is a side view of an aortic tool.

FIG. 4B is a cross-section view of the aortic tool shown in FIG. 4A.

FIG. 4C is a side view of an aortic tool.

FIG. 4D is an enlarged view of the distal end of the aortic tool shown in FIG. 4B

FIG. 5A is a side view of a ventricular tool.

FIG. 5B is an enlarged view of the distal end of the ventricular tool.

FIG. 5C is a cross-sectional view of FIG. 5B.

FIG. 5D is an enlarged view of the distal end of the ventricular tool.

FIG. 5E is a cross-sectional view of FIG. 5D.

FIGS. 6A-G are a series of combination side views and cross-sectional views of the aortic tool and aorta showing the aortic connection insertion steps.

FIGS. 7A-F are a series of combination side views and cross-sectional views of the ventricular tool and heart showing the ventricular connection insertion steps.

FIG. 8 shows an implant with a protective coil over the conduit.

FIGS. 9A and 9B shows a mixed side view/cross sectional view of an implant with one slit used for both an aortic and a ventricular tool.

### DEFINITIONS

The terms “ventricular” or “ventricular end” and “aortic” or “aortic end,” when used herein in relation to the implant devices or tools location or direction, refer to the end of the device or tool or the direction nearest the ventricle or nearest the aorta, respectively.

The terms “proximal” and “distal” when used herein in relation to instruments used in the procedure of the present invention, respectively refer to directions closer to and farther away from the operator performing the procedure.

### DETAILED DESCRIPTION OF THE INVENTION

This invention reveals the implant, tools, and method required to safely create a one-way blood pathway through a heart chamber wall to a blood vessel without stopping the heart or occluding the blood vessel.

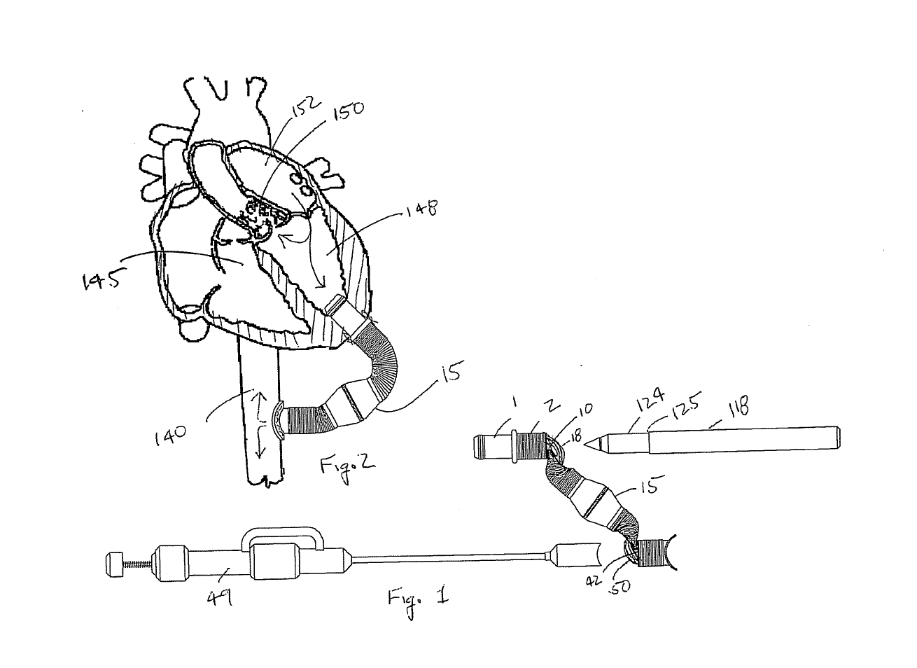

Generally described, the implant consists of a hollow conduit with a one-way valve located within the conduit as shown in FIG. 1. Between the valve and each end of the conduit is a slit opening in the conduit. The two slit openings allow insertion of heart and vessel cutting tools into and out of either end of the conduit without damaging the valve located between the slits. If there was only one slit, internal access to both ends of the conduit could only be obtained by going through the valve. To minimize blood loss, the cutting tools inserted through the slits fit snugly within the conduit. Also to minimize blood loss, the slit openings are designed for rapid repair at the end of the procedure. To improve flow, the conduit is tapered to allow the more flow resistant heart valve to be larger in diameter than the ends of the conduit. To improve the procedure, ventricular access tools have been invented to allow connection to a beating heart without removing tissue or loosing excessive blood. Aortic access tools have been invented to allow a high flow connection to a pressurized blood vessel without clamping the vessel, without occluding flow within the vessel or loosing excessive blood. These and other features of the invention are described herein.

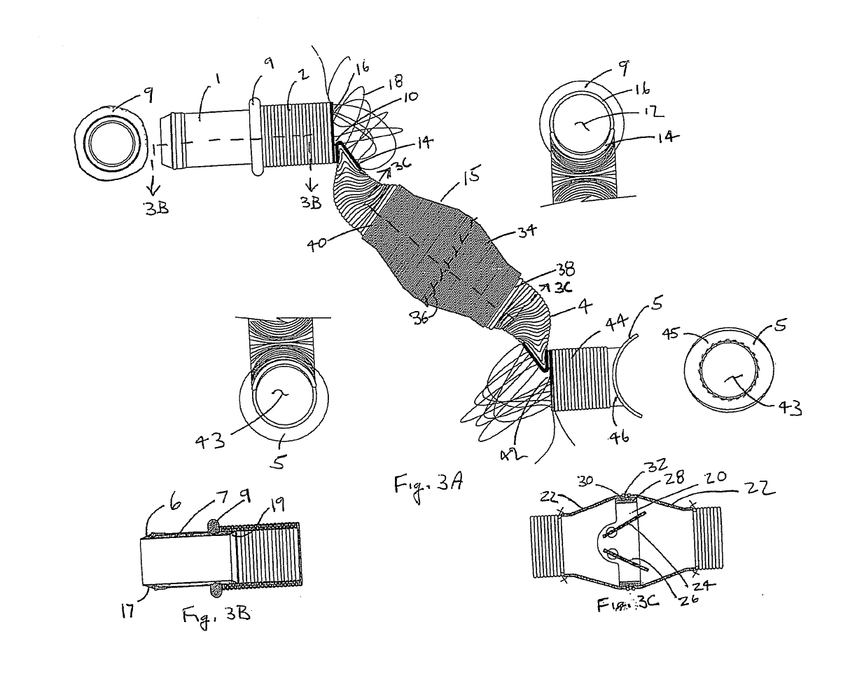

FIG. 2 shows the implant 15 connected between the Ventricle 148 and the Aorta 140 in a heart 145 with a stenotic, calcified native aortic valve 150. In this position, blood flowing from the left atrium 152 to the left ventricle 148 can be ejected out of the ventricle 148 by either flowing through the stenotic native aortic valve 150 or through the implant 15. Once blood is in the aorta 140, it can flow either to the brain, heart, and upper extremities or to the liver, kidney, and the lower extremities.

Description of Invention Structure

Valve/Conduit Implant

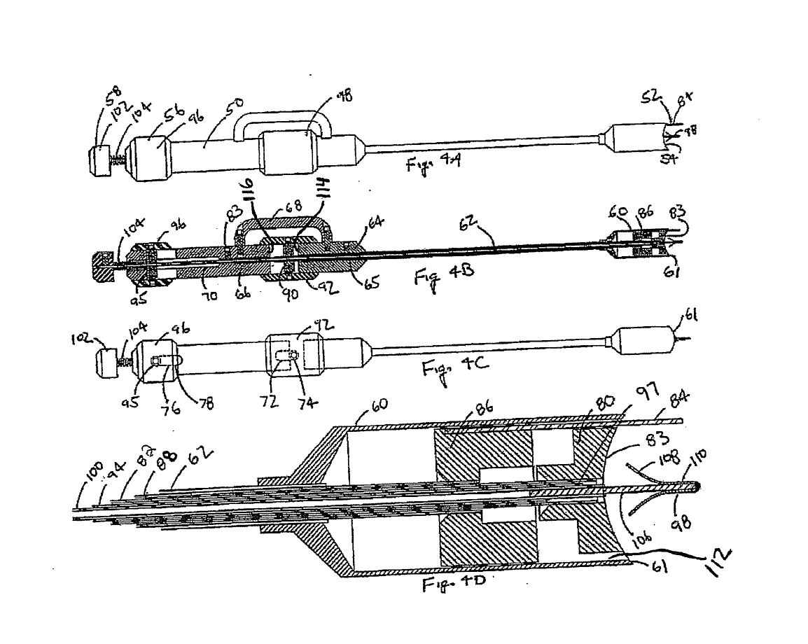

A preferred embodiment of a Valve/Conduit Implant 15 is shown in FIGS. 3A-C. The implant is comprised of five main assemblies as shown in FIG. 3A; a Ventricular Connector 1, a Ventricular Conduit Segment 2, a Valve Segment 3, a Aortic Conduit Segment 4, and an Aortic Cuff 5. The assemblies are integrated into one implant device called the Valve Bypass Conduit. The following is a more detailed description of each assembly.

The Ventricular Connector 1 is composed of two elements as shown in cross section in FIG. 3B; a Ventricular Orifice 6 and a Ventricular Cuff 7. The Ventricular Orifice 6 is cylindrically shaped and composed of a rigid biomaterial material such as stainless steel, titanium, or pyrolytic carbon. The internal diameter of the Ventricular Orifice in a preferred embodiment would be sized to have a cross sectional area of about 2 square centimeters, which equates to a diameter of about 16 millimeters. Depending on flow requirements, the diameter could vary between 13 and 25 millimeters. The outside diameter of the ventricular end of the Orifice 6 has an insertion taper or insertion chamfer 17 and the inside diameter of the aortic end has a flow taper 19 to facilitate flow. The Ventricular Cuff 7 is made of polyester or some other conventional biomaterial and covers the exterior surface of the Orifice 6 except for the tapered surface 8 on the ventricular end. Near the aortic end of the Orifice 6, the Cuff 7 is gathered to form a Sewing Ring 9. The Ventricular Connector Segment 1 is connected to the Ventricular Conduit Segment 2.

The Ventricular Conduit Segment 2, shown in side view in FIG. 3A and in cross section view in FIG. 3B is composed of knitted polyester or some other conventional biomaterial graft material such as Teflon or bovine arterial tissue with an inside diameter consistent with the inside diameter of the Ventricular Connector. Near the middle of this Conduit Segment 2, the conduit material is cut or sliced along about 5/9ths of the perimeter of the conduit to form a Ventricular Slit 10. As shown in FIG. 3A, the slit 10 allows the conduit to be inverted to allow for full cross sectional access to the interior lumen 12 of the conduit 2. The exposed edges 14 and 16 are hemmed with a Polyester Thread or Suture to slightly reinforce the edge and to minimize fraying. A similar Polyester Suture 18 is then sewn loosely between the cut edges, alternating from edge to edge. This continuous suture is not immediately tightened to form co-adaptation of the edges, but is left loose to allow subsequent insertion of tools equal in diameter to the Ventricular Conduit Segment 2.

The Valve Segment 3 of the Implant, as shown in cross section in FIG. 3C, comprises a prosthetic valve 20 enclosed within a Double Tapered Conduit 22. The valve 20 in a preferred embodiment is sized to have an effective orifice area of about 2.5 square centimeters. Effective orifice area is a conventional descriptor of valve flow performance use by all valve manufacturers. One such valve meeting this Effective Orifice Area requirement is a 23 mm St. Jude Medical Regent pyrolytic carbon valve with its standard attachment cuff removed. This valve type, known to those knowledgeable in the art as a bi-leaflet valve, is comprised of two Leaflets 24 and 26 captured within an Orifice Ring 28. The Ring has a Groove 30 formed on its exterior surface. The conduit portion of this segment is double tapered to allow both ends of the Double Tapered Conduit 22 to be compatible with the adjoining Conduit Segments and to also allow the enlarged middle section to be compatible in diameter with a the valve Orifice Ring 28. The transition in diameters is gradual to maintain smooth flow dynamics. This transition in conduit diameter can be done by stretching the conduit material or cutting a pattern and then sewing the material into two opposed funnel-like shapes. The Valve 20 is attached to the Double Tapered Conduit 22 by wrapping a Suture 32 tightly around the Double Tapered Conduit 22 over the Groove 30 in Orifice Ring 30 thereby forming an interference fit. Fitted over the Double Tapered Conduit 22 is a tightly wound Support Coil 34 as shown in FIG. 3A. The Support Coil 34 is composed of Nitinol, a shape memory metal with super-elastic properties. The Support Coil 34 is attached to the center of the Conduit by Sutures 36 or some other suitable attachment method. The Aortic End 38 and Ventricular End 40 of the Support Coil 34 are not attached to the Conduit and are free to be stretched or extended.

The Aortic Conduit Segment 4, as shown in FIG. 3A, is similar in design and composition to the Vascular Conduit Segment 2. An aortic slit 42 is formed in a Conduit 44 through about 5/9ths of its perimeter. The slit 42 allows the conduit to be inverted to allow for full cross sectional access to the interior lumen 43 of Conduit Segment 2. The exposed edges 46 and 48 are hemmed. A loosely sewn Suture 50 is sewn between the edges 46 and 48 such that a tool sized similar to the internal diameter of Conduit 44 could be passed through an opening in the Suture 50 and then advanced into the Conduit 44. If this suture 50, like the one place in the Ventricular Conduit Segment 2, is pulled tight, the hemmed edges 46 and 48 of the Slit 42 will move into direct opposition to form a tight fighting joint, essentially repairing the Slit 42 in the Conduit 44. Again, similar to the Ventricular Conduit Segment 2, The Aortic Valve Segment 4 is sewn to the Valve Segment 3. The other end of the Aortic Conduit Segment 4 is cut in a circular arc to form a curvilinear edge 46 similar in radius to the radius of an aorta.

Attached to the aortic end of the Aortic Conduit Segment 4 is the Aortic Cuff 5. The Cuff 5 is formed of a polyester fabric shaped into a ring. The internal edge 48 of Cuff 5 is attached to the curvilinear edge 46 of Conduit 44 by sewing or some other conventional method such that the Cuff 5 forms a blood tight seal and extends radially outward from the Conduit 44 to form a mating surface compatible with the surface of the aorta The aortic surface 45 of Cuff 5 is intended to lay flat against the Aorta.

Aortic Anastomotic Tool

A preferred embodiment of an Aortic Tool 49 is shown in FIG. 4A-C. The device is composed of five assemblies: a Handle Assembly 50, a Cutting Assembly 52, a Contour Positioning Assembly 54, a Piercing Assembly 56, and an Anchor Assembly 58. The following is a description of each assembly.

The Handle Assembly is composed of a hollow cylindrical Shroud 60 connected to a Handle Cannula 62. The opposite end of Cannula 62 is connected to a Distal Handle Body 64. The distal end of the cylindrical Shroud 60 is cut to form a contoured edge 61 to make edge contact with the Aorta when the Tool is positioned against the Aorta The Distal Handle Body 64 has a Central Lumen 65. The distal end of Lumen 65 is sized to allow insertion and bonding of the Handle Cannula. The Distal Handle Body 64 is stationary and coaxial relative to a Proximal Handle Body 66 by common connection to a Bridge Connector 68. The Bridge Connector 68 separates the two handle bodies by a fixed distance. The Proximal Handle Body 66 has a Central Lumen 70 sized to fit other cannula in the tool as will be explained later. At its distal end. the Proximal Handle Body 66 has a Slot 72 sized to fit a pin 90 described later. Near its proximal end. the Proximal Handle Body 66 has two Slots 76 and 78 cut into the wall formed by the Central Lumen 70. These slots are spaced 180 degrees apart. The slots are cut in the direction of the main axis of the body.

The Contour Positioning Assembly 54 is composed of a Contour Positioning Hub 80 connected to a Contour Positioning Cannula 82. Like Shroud 60. the Distal Surface 83 of Contour Positioning Hub 80 is formed to allow surface contact across the entire surface of the Aorta when the tool is positioned perpendicular to the Aorta.

The Cutting Assembly 52 is composed of a Cutting Blade 84 attached to a cylindrical Cutting Hub 86. The Cutting Hub 86 is attached to one end of a Cutting Cannula 88. The other end of the Cannula lies within a Coaxial Hole located at the center of Cutting Knob Pin 90. Set screws threaded into both ends of a transverse hole in the Cutting Knob Pin 90 securely engage the Cannula 88 to the Cutting Knob Pin 90. The Cutting Knob Pin 90 fits into holes located 180 degrees apart on a Cutting Knob 92. The Cutting Knob 92 is ring shaped with an internal diameter sized to have a sliding fit with both the Distal Handle Body 64 and the Proximal Handle Body 68.

A Piercing Assembly 56 consists of a Piecing Cannula 94 connected to a Piercing Knob 96 in similar fashion as the Cutter Assembly 52 just described. The proximal end of the Piercing Cannula 94 lies within a Coaxial Hole located at the center of a Piercing Knob Pin 95. Set screws threaded into both ends of a transverse hole in the Piercing Knob Pin 95 securely engage the Cannula 94 to the Piercing Knob Pin 95. The Piercing Knob Pin 95 fits into holes located 180 degrees apart on the Piercing Knob 96. The Piercing Knob 96 is ring shaped with an internal diameter sized to have a sliding fit with the Proximal Handle Body 66. The opposite end of the Piercing Cannula 94 is angle cut to faun a sharp tip 97 similar to that on a hypodermic needle.

The Anchor Assembly 58 is composed of an Anchor Tip Assembly 98, an Anchor Cannula 100, an Anchor Knob 102, and an Anchor Spring 104. The Anchor Tip Assembly 98, as shown in detail in FIG. 4C, is composed of a Core Wire 106 centered around six Wires or Tines 108. The exact number of tines is not important, any number between two and twelve could be used. The central Core Wire 106 and the surrounding Tines 108 are encased in a tight fitting End Cap 110. The Tines are plastically deformed in a radial outward fashion to form curvilinear shapes. The Anchor Spring 104 is inserted over the Anchor Cannula 100 and then the Anchor Knob 102 is attached to the free end of the Anchor Cannula 100.

The five assemblies just described are logically placed in a coaxial fashion as described below to create the Aortic Tool.

The Anchor Cannula 100 fits within the Piercing Cannula 94 such that when the Anchor Spring 104 is in light contact with both the Anchor Knob 102 and the Piercing Knob Pin 95 the distal tip of the compressed Anchor Assembly 58 resides just proximal of the proximal edge of the angle cut distal end of the Piercing Cannula 56.

The Piercing Cannula 94 fits within the Contour Positioning Cannula 82. The Piercing Knob Pin 95 fits into the Proximal Slots 76 and 78 of Proximal Handle Body 66. When the Piercing Knob 96 is positioned in its most proximal position, the tip of the Piercing Cannula 94 lies just proximal of the distal opening on the Contour Positioning Hub 80.

The Contour Positioning Cannula 82 fits within the Cutting Cannula 88. The proximal end of the Contour Positioning Cannula 82 is attached to the Proximal Handle Body 66 using Set Screw 83. The proximal end of the Cutting Cannula 88 is attached to the Cutting Knob 92 through an interference fit with Cutting Knob Pins 90. The rotatable Cutting Hub 86 resides proximal of the stationary Contour Positioning Hub 80.

The Cutting Cannula 88 fits within the Handle Cannula 62. When the Cutting Knob Pin 90 resides in it most proximal position within the distal slot 72 of Proximal Handle Body 66, the distal tip of the Cutting Blade 84 resides just proximal of the most distal edge of the Shroud 60. Set Screw 83 is loosened to allow Contour Cannula 82 to be rotated until its distal end Contour Surface 83 perfectly aligns with the contour on the Shroud 60. Set Screw 83 is the tightened.

The Shroud 60 and Contour Positioning Hub 80 do not move relative to each other because the Shroud 60 and Contour Hub 80 are connected, respectively, to the Distal 64 and Proximal Handle Bodies 66 that, in turn, are connected to each other through the Bridge Connector 68. It can therefore be appreciated that the Cutting Blade 84, connected by the Cutting Cannula 88 to the Cutting Knob 96, can be advanced through the annular slot 112 formed between the stationary Shroud 60 and Contour Positioning Hub 80. The movement of the Cutting Blade 84 in the axial direction is limited by the travel of the Cutting Knob Pin 90 within the gap between the proximal surface 114 of the Distal Handle Body 64 and the distal surface 116 of the Proximal Handle Body 66.

Ventricular Tool

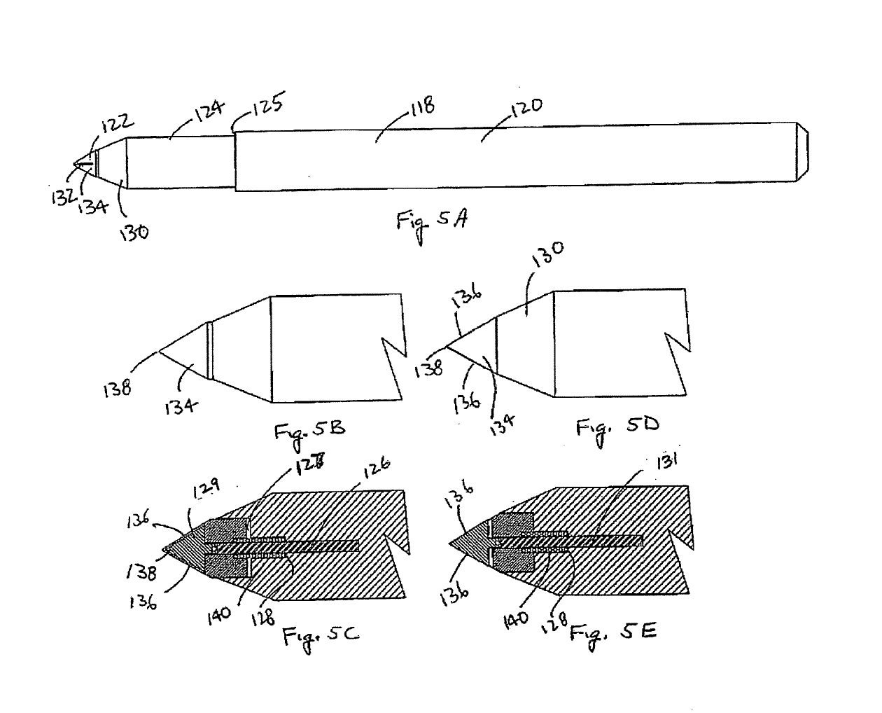

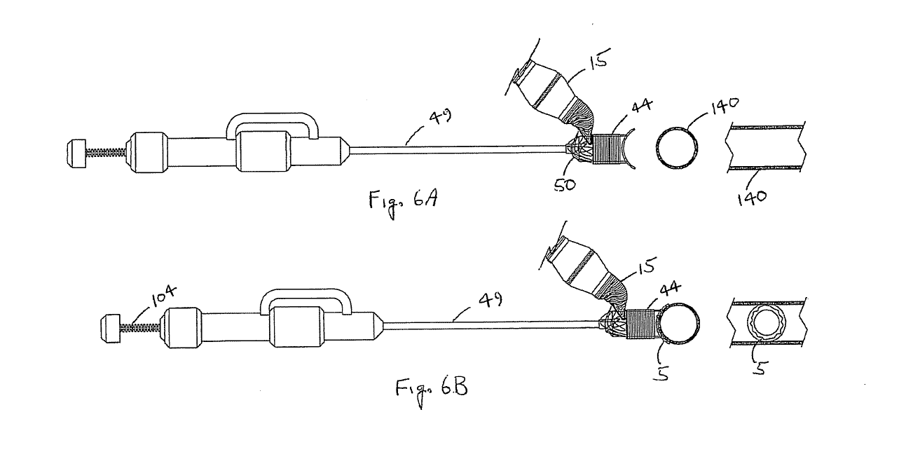

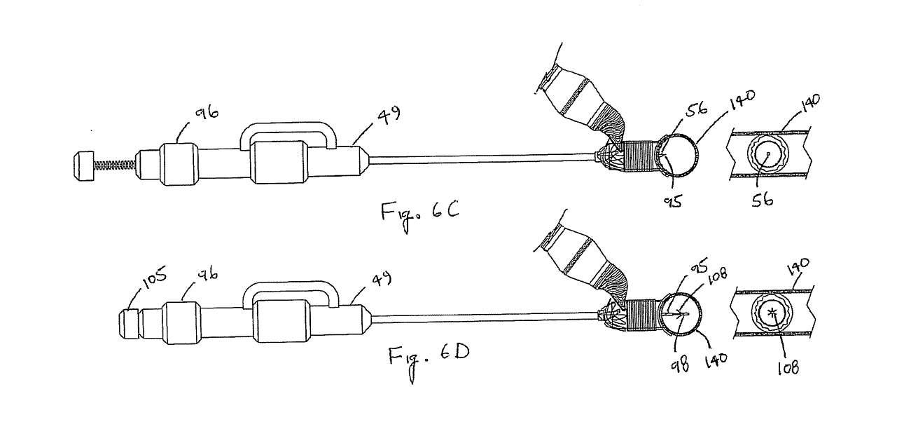

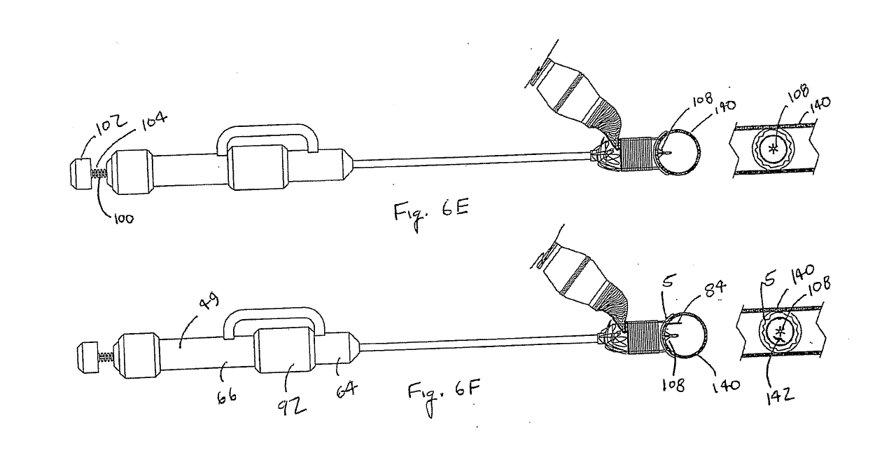

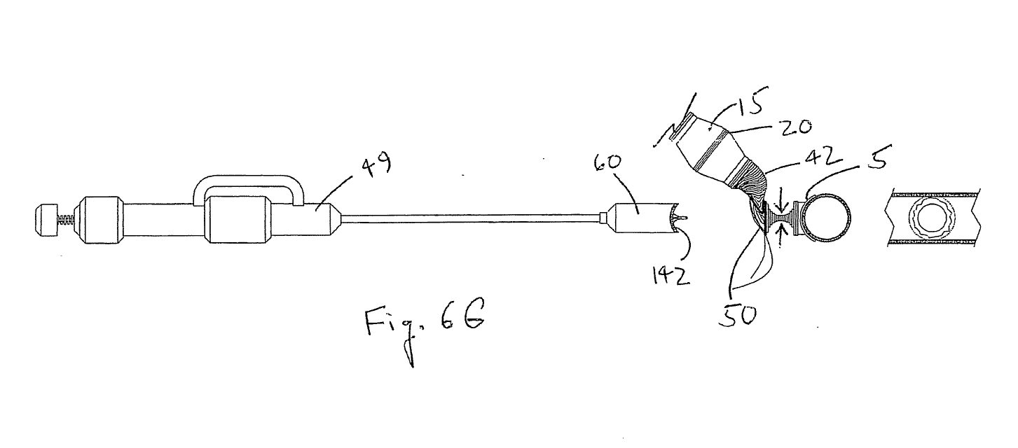

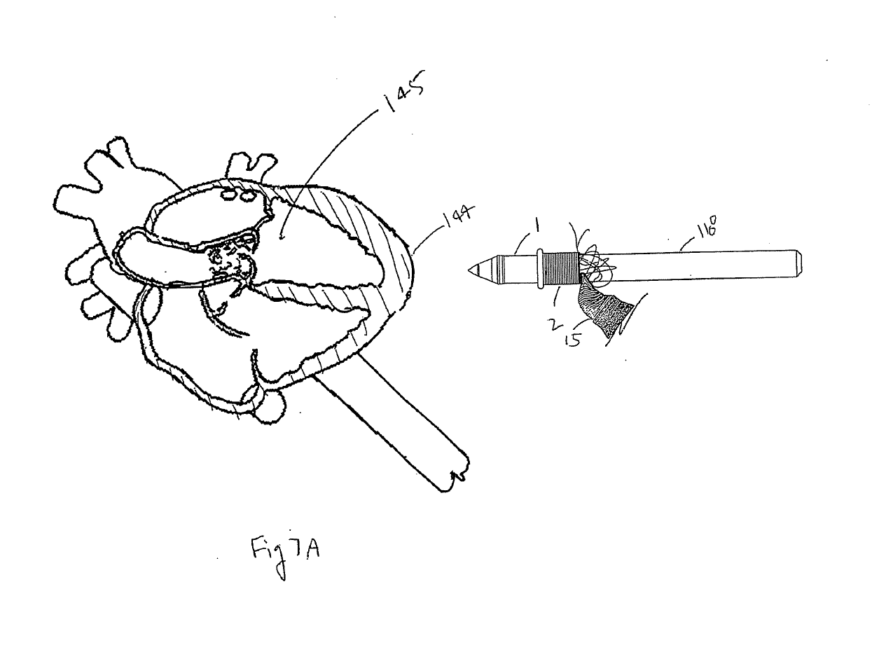

A preferred embodiment of a Ventricular Tool 118 is shown in FIG. 5A-E. The device is composed of a Body Component 120 and a Cutting Assembly 122. The following is a description of each assembly.

The Body 120 is generally cylindrical in shape. The Body 120 is sized so the internal diameter of the Ventricular Connector 1 fits closely to the external diameter of the distal section 124 of the Body 120. A taper or chamfer 125 is located between the distal section 124 and the rest of the Body 120. The Body 120 has an internal lumen 126 starting at its distal end extending coaxially into the Body. The lumen has a countersink feature forming an internal edge 128. When the mating Ventricular Connector 1 is inserted over the Body 120, a chamfered surface 130 on the distal end of the Body 120 forms a near flush smooth transition between the two components. This smooth transition is important when inserting the two devices simultaneously into tissue. The proximal end of the device is sized to be comfortably held in the hand of a surgeon.

The Cutting Assembly 122 is composed of a protected Cutting Blade 129 attached to a Cutting Shaft 131. The other end of the Cutting Shaft 131 is attached to the Body 120 by bonding or otherwise affixing to its internal lumen. The Cutting Blade 129 is positioned within a Slit 132 in a Protective Slotted Tip 134. The Protective Slotted Tip 134 is tapered to a blunt tip on its distal end. The Protective Slotted Tip 134 protects the Cutting Edges 136 of Blade 129 when there is no insertion pressure. When the tapered tip 138 of the Protective Slotted Tip 134 is pressed axially against a surface, the Protective Slotted Tip 134 slides proximally over the Cutting Shaft 131 by compressing Spring 140 against the internal edge 128 of Body Lumen 127 thereby exposing the Blade Edges 136. When axial tip pressure is relieved, the Spring 140 acts to slide the Protective Slotted Tip 134 forward relative to the Cutting Shaft 131 and Blade 129 to re-encase or protect the Blade Edges 136.

Operation

Initially, access is made through the chest cavity to expose the left ventricle including the apex of the heart and the descending aorta. Once exposed, a suitable location is identified on the aorta to create the aortic connection.

Connection to Aorta

As shown in FIG. 1, the Aortic Tool 49 is inserted into the Implant 15 by entering through the aortic slit 42 between two adjacent loosely sewn loops of Suture 50. The outside diameter of the Aortic Tool 49 is slightly undersized relative to the inside diameter of the Conduit 44 and Aortic Cuff 5 to allow passage yet minimize blood flow.

As shown in FIG. 6A, the distal Shroud 60 of Aortic Tool 49 is axially and rotationally positioned relative to the Aortic Cuff 5 such that the contour edge 61 of Shroud 60 and the associated Contour Hub distal surface 83 are aligned with and flush with the similar contour surface 45 of the Aortic Cuff 5.

Once properly aligned, the Aortic Tool 49 and Aortic Cuff 5 are positioned as one unit against the target site on the Aorta 140 as shown in FIG. 6B. While maintaining this position, the surgeon attaches the Aortic Cuff 5 to the Aorta 140 using conventional sutures and suturing techniques.

The combination of a close fitting Aortic Tool 49 within the Conduit 44 and the Aortic Cuff 5 already sutured in place allows the creation of a fluid connection between conduit 44 and aorta 140 without disturbing blood flow, without applying any clamping forces to the aorta 140, and without excessive blood loss. This unique connection method is completed as follows.

First, as shown in FIG. 6C, the Piercing Cannula 56 contained within the Aortic Tool 49 is advanced into the aorta 140 by fully advancing the Piercing Knob 96 forward. This action places the Tip 95 of the Piercing Cannula 56 into the aorta 140. The stroke distance is limited to ensure that the Piercing Cannula Tip 95 does not advance across the aorta 140 to cut or puncture the other side of the aorta.

Next, as shown in FIG. 6D, the Anchor Assembly 98 is advanced out of the tip 95 of Cannula 56 by advancing the Anchor Knob 102 fully forward, compressing Spring 104. This action radially deploys the Tines 108.

Next, as shown in FIG. 6E, the Anchor Knob 102 is released, allowing Spring 104 to relax causing the Anchor Cannula 100 to move proximally that, in turn, allows the expanded Tines 108 to lodge securely into the aorta 140. The axial compressive force produced by the Spring 104 keeps the Tines 108 in close contact with the aorta 140.

With the Tines 108 deployed and pressing against the aorta 140 and the Aortic Cuff 5 securely sutured to the aorta 140, the Cutting Blade 84 is advanced through the aorta 140 by advancing the Cutting Knob 92 forward as shown in FIG. 6F. Once advanced, the Knob 92 is rotated in a circular fashion to begin radially excising a piece of aortic wall 142. To facilitate the cutting action, the Cutting Knob 92 can also be actuated by the surgeon in a back and forth method a fixed distance between the proximal surface of the Distal Handle Body 64 and the distal surface of the Proximal Handle Body 66 as it is rotated to give the Cutting Blade 84 a slicing or sawing action. The rotation and axial sawing action is continued until the Cutting Knob 92 and connected Cutting Blade 84 have been rotated a minimum of 360 degrees. At this point, aortic wall tissue 142 about the size of the inside diameter of the Aortic Cuff 5 is fully excised and separated from the wall of the aorta 140 and is firmly attached to the Aortic Tool 49 through means of the deployed Tines 108.

Next, as shown in FIG. 6G, the Aortic Tool 49 with attached excised aorta wall 142 is carefully retracted until the proximal end of Shroud 60 is seen emerging from the aortic slit 42 in the Conduit 44. At this point, the blood filled conduit segment between the Shroud 60 and the Aortic Cuff 5 is externally occluded using a standard vascular clamp or other such conventional method. With Conduit 44 occluded, the Aortic Tool 49 is removed through first the aortic slit 42 and then through the loosely sewn Suture 50. The Suture 50 is then tightened, causing the edges of Slit 42 to move together in close apposition to form a blood tight repair. The Suture 50 is knotted securely and the Clamp is removed. The One-Way Valve 20 located within the conduit prevents any significant blood flow exiting the Implant 15 since the aortic blood pressure maintains the valve in the closed position.

With the aortic connection now complete, the next step is to connect the ventricular end of the Implant to the Left Ventricle.

Connection to Ventricle

As shown in FIG. 1, the Ventricular Tool 118 is inserted into the Ventricular Connector land Conduit Segment 2 of Implant 15 by entering the Ventricular Slit 10 between two adjacent loosely sewn loops of Suture 18. The outside diameter of the distal portion 124 of Ventricular Tool 118 is slightly undersized relative to the inside diameter of the Conduit 2 and Ventricular Connector 1 to allow passage of the tool yet form a nearly blood tight sliding seal. The Ventricular Tool 118 is advanced into the Implant 15 until its chamfer 125 abuts the Chamfer 19 on the Ventricular Connector. With the Chamfers abutting, the two components will move as one if an axial force is applied to the Ventricular Tool 118 in the ventricular direction. Conversely, if the force is applied in the aortic direction, the Ventricular Tool 118 can separate from the Ventricular Connector 1.

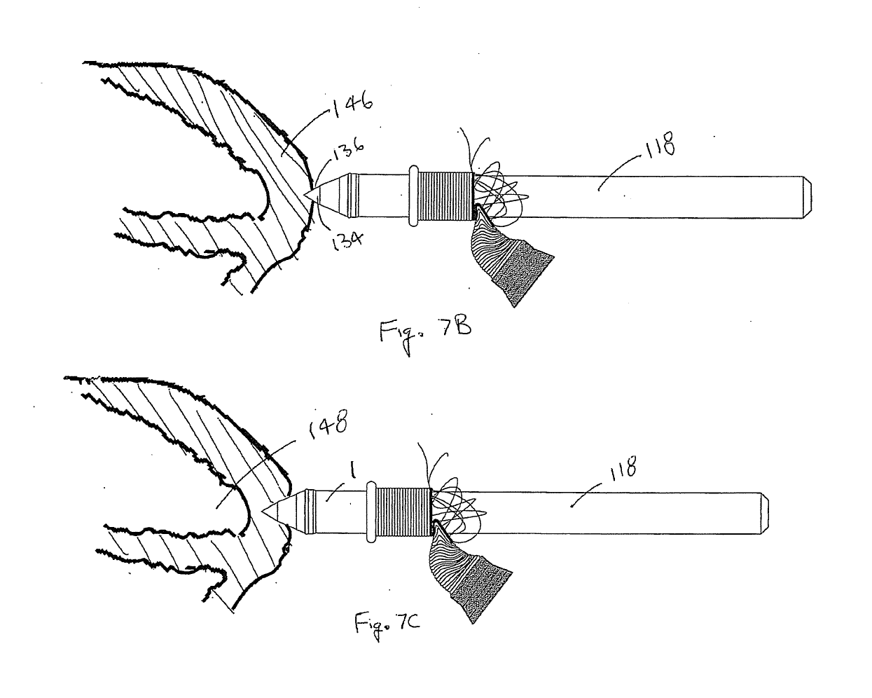

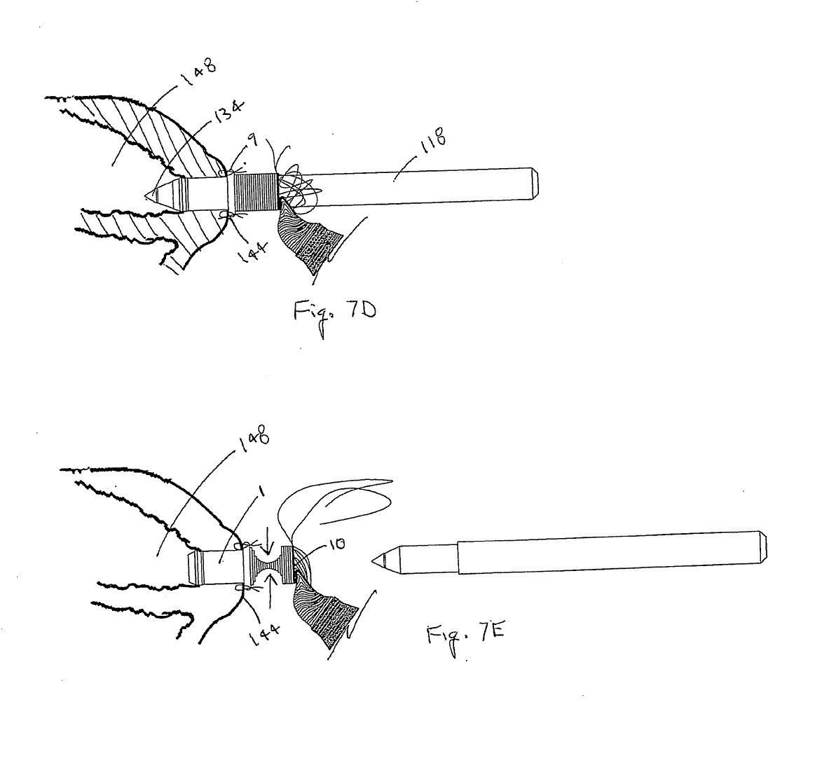

With tool and implant ready, a suitable location on the epicardial surface 144 of the heart 145 is identified at or near the left apex as shown in FIG. 7A. At the selected site, the tip of the Ventricular Tool 118 is pressed against the heart wall as shown in FIG. 7B. The axial component of the pressing force acts to compress the Shroud Spring thereby sliding the Shroud proximally to expose the Cutting Edges 136 of the Cutting Blade. The sharp Cutting Edges cut into the myocardium wall 146 allowing the tapered Protective Shroud 134 to advance into the myocardium as shown in FIG. 7C. Due to the Shoulder/Chamfer abutting fit, the Ventricular Connector I moves into the Ventricle 148 simultaneously with the Ventricular Tool 118. The Cutting Blade only cuts the heart myocardium in contact with it. The myocardium tissue that is not cut by the blade is dilated by the advancing tapered surfaces of the combined Protective Shroud 134 and Ventricular Connector 1. When the Protective Shroud 134 of the Ventricular Tool 118 emerges into the ventricle 148 as shown in FIG. 7D, the Protective Shroud 134 is no longer exposed to an axial force from the heart myocardium and the Compressed Spring located within the Ventricular Tool 118 acts on the Shroud to re-advance the protective Shroud distally to again cover the Cutting Edges of the Cutting Blade. With Shroud advanced distally, the sharp Cutting Edges cannot damage any structures in the moving ventricle. Insertion continues until the Ventricular Connector Cuff 9 abuts the epicardial surface 144 of heart 145. Due to the sufficiently long length of the Ventricular Connector 1, when the Cuff 9 abuts the epicardial surface 144, the entrance to the Ventricular Connector 1 is fully exposed to the Ventricle.

The Cuff is then sutured to the Ventricle wall using conventional suturing techniques to faun a blood tight seal.

After the Cuff 9 is securely attached to the heart surface 144, the Ventricular Tool 118 is carefully retracted until it is completely removed from the Ventricular Connector portion of the implant as shown in FIG. 7E. At this point, the blood filled conduit segment between the tip of the Ventricular Tool and the Ventricular Connector is externally occluded using a standard vascular clamp or other such means. With Conduit 2 occluded, the Ventricular Tool 118 is removed through first the Ventricular Slit 10 and then through the loosely sewn Suture 18. The Suture 18 is then tightened causing the edges 14 and 16 of the Slit to move together in close apposition to form a blood tight repair. Before completely tightening the suture, any air within the conduit is vented using conventional surgical techniques. The Suture 18 is then completely tightened and securely knotted. The Clamp is removed. The Implant 15 is shown in FIG. 7F installed between the ventricle 148 and the aorta 140.

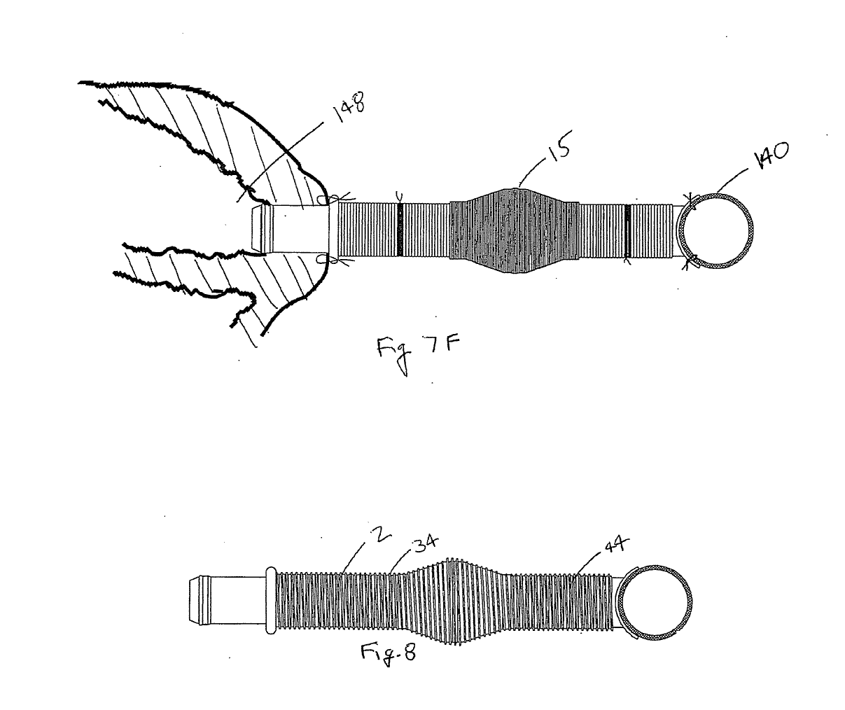

Once installed and all connections and functions are verified, the Support Coil 34 is expanded to cover the Ventricular Conduit 2 and the Aortic Conduit 44 as shown in FIG. 8.

To complete the procedure, the chest incision is closed according to standard technique.

Summary, Ramifications, and Scope

The reader will see that the invention, consisting of an implantable device, two implant tools, and a new method, alleviates the prior art problems associated with off-pump apicoaortic procedures.

The invention, when compared to prior art, minimizes the potential damage to the ventricle, minimizes blood loss during the beating heart procedure, improves the aortic connection method, minimizes potential damage to the aorta and the associated potential of generating emboli, improves blood flow through the implant, and allows the surgeon to perform the procedure quicker, easier, and more predictably.

Specifically, the invention has the following advantages:

It minimizes potential damage to the heart:
by not removing any myocardial tissue when creating a new arterial connection to the left ventricle of a beating heart. This is accomplished by employing a ventricle access tool that pierces and dilates the myocardium tissue sufficient only to allow tool and conduit access, but does not core or otherwise remove any myocardial tissue.
by preventing inadvertent cutting of any nearby ventricular endocardial surfaces or structures when creating a connection to the left ventricle of a beating heart. This is accomplished by having a spring-loaded protective shroud on the end of the ventricular tool that encases the sharp cutting edges on the tool once the tool has entered the ventricle space.

It allows for connection of a conduit to a fully pressurized aorta:
without squeezing or clamping the aorta, an action known to damage the aorta or dislodge emboli.
without stopping or reducing blood flow through the aorta.
with an improved connection technique whereby the conduit is sewn to the aorta before the aorta is cut. This is facilitated by having a pre-formed cuff on the conduit that fits to the aorta's cylindrical surface like a horse saddle fits to the back of a horse.
without damaging the far wall of the aorta during cutting or loosing the excised piece of tissue within the aorta by employing an innovative hole cutting tool that incorporates an intra-luminal anchor in combination with a limited stroke cutting blade rotated around a stationary shroud shaped to conform to the cylindrical surface of the exposed aorta.

It minimizes blood loss when connecting the conduit to either the aorta or ventricle wall
by using a conduit with an aortic connection cuff that allows the surgeon to sew the cuff to the aorta before the aorta is cut open so that when the aorta is cut open, the blood entering the conduit will not leak at the connection site.
by incorporating slits in the conduit that allows insertion or removal of the conduit occluding aortic or ventricular tools into or out of the conduit without interfering with the one-way valve located between the two slits.
by employing aortic and ventricular cutting tools that fit snugly within the conduit such that blood entering the conduit at the connection site cannot leak past the tool and out the other end of the conduit.
by having a loosely sewn suture looped between both edges of each slit in the flexible conduit so that a rapid, blood tight repair of each slit can be made by tightening and tying the suture immediately after each conduit occluding tool is removed.

It maximizes blood flow through the implant
by using an innovative aortic cutting tool that cuts a side-hole in the aorta that matches the internal diameter of the conduit.
by employing a one-way valve that is larger in diameter than the conduits connected to it. The larger valve size compensates for the inherent flow restriction due to the valve components residing within the flow stream.
by preventing the flexible conduit from kinking if it is formed in a tight radius or if an external crushing force assaults it. This is accomplished by installing a super elastic alloy coil around the conduit which provides radial strength and kink resistance without effecting the inherent flexibility, biocompatibility, or compliance of the conduit.

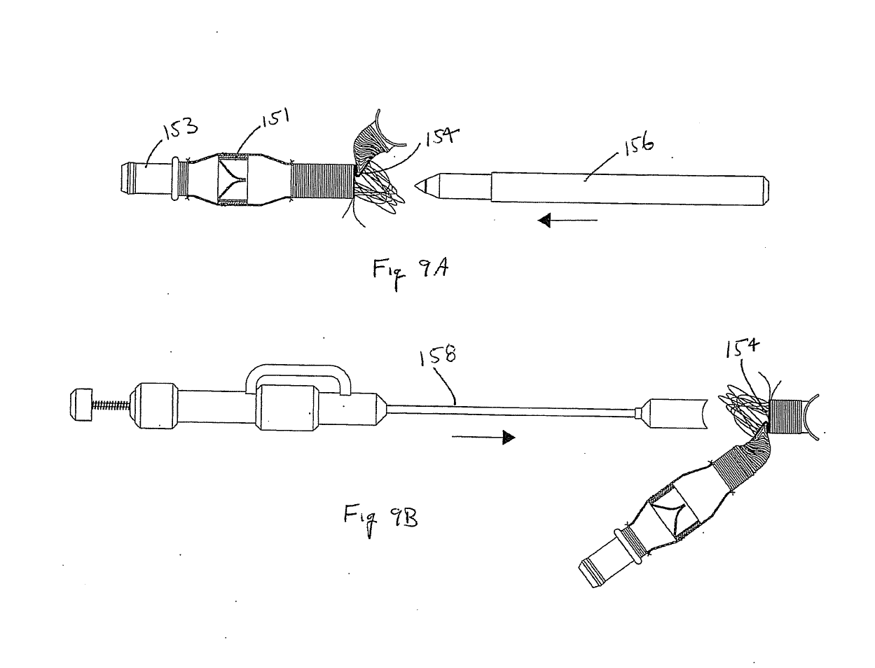

Although the description above contains many specifications, these should not be construed as limiting the scope of the invention but as merely providing illustrations of the presently preferred embodiment of this invention. For example:
Only one slit need be employed if access to only one connection is deemed sufficient.
A cylindrical shaped cutting blade, similar to a cork borer or apple corer, could be employed as the aortic wall cutting tool.
The order of operation could be switched to do the ventricular connection first, than the aortic connection.
Reference was made throughout the application that the aorta is the specific vessel connection site. Other vessel locations, as described by Cooley and others in prior art, would be just as feasible. Also, other locations in the heart, such as the right ventricle, could be accessed as well, as described in prior art.
The valve diameter could be sized to fit within a constant diameter conduit similar to prior art.
Other slit closing means could be used such as staples or clips. Also, hoops composed of Nitinol or some other elastic material could be sewn into the opposing edges of the slits to facilitate a more rapid closure.
The aortic connection is shown generally perpendicular to the aorta in the embodiment described. The connection could be made at a more slanted angle to facilitate blood flow coming from the conduit up towards the brain and coronaries.
The proximal surface of the Distal Handle Body and the distal surface of the Proximal Handle Body can be spaced apart from each other at different distances to allow for different cutting blade stroke lengths. Also, the surfaces need not be perpendicular to the Cutting Knob lumen such that as the Cutting Knob is rotated, the cutting blade stroke length and insertion distance can be varied.
The length and diameter of the implant can be made to any desired dimension.
The size of the valve can be any size deemed acceptable with regard to flow and can be chosen independent of the size of the connecting conduits.
Other bioprothesis valves, either of carbon, tissue. polymer or other conventional construction, could be easily substituted in place of the particular St. Jude Medical Regent carbon valve selected in this embodiment of the implant invention.
The aortic conduit segment can be cut to a desired length by the surgeon and then sewn or otherwise attached to the aortic connector before implantation.
FIGS. 9A and 9B show a single slit embodiment of the invention. The Valve 151 is placed between the Ventricular Connector 153 and the Ventricular Slit 154 such that a Ventricular Tool 156 can be carefully inserted through the Valve 151 to access the ventricle. As shown in FIG. 9B, the Ventricular Slit 154 could also be used to insert an Aortic Tool 158 into the aorta.

Thus, the scope of the invention should be determined by the appended claims and their legal equivalents rather than by the examples given.

What is claimed is:

1. A medical implant comprising:
a hollow conduit having a first end opening, a second end opening, and a slit opening located between said first and second end openings; and
a one way valve located within said conduit.

2. The medical implant of claim 1, wherein said slit opening is defined by a pair of opposing edges.

3. The medical implant of claim 2, further comprising a closing means associated with said slit opening that is structured to secure said pair of opposing edges adjacent one another, wherein closing said slit opening with said closing means provides for a closed path between said first end opening and said second end opening of said conduit.

4. The medical implant of claim 3, wherein said closing means comprise a suture.

5. The medical implant of claim 4, wherein said pair of opposing edges are hemmed edges, and wherein said sutures are sewn through said hemmed edges.

6. The medical implant of claim 5, wherein said sutures are formed from polyester.

7. The medical implant of claim 3, wherein said closing means comprises one or more staples.

8. The medical implant of claim 3, wherein said closing means comprises one or more clips.

9. The medical implant of claim 3, wherein said slit opening extends through about 5/9ths of a perimeter of said conduit.

10. A method for creating a one-way blood pathway through a heart chamber wall to a blood vessel, the method comprising:
a) selecting a medical implant comprising a hollow conduit having a first end opening, a second end opening, a one-way valve located between said end openings biased to allow one-way flow from said second end opening to said first end opening, and a slit opening located between said second end opening and said one-way valve;
b) identifying a location on said blood vessel to connect said first end opening of said conduit;
c) selecting a vessel wall cutting tool sized to fit through said slit opening and said first end opening;
d) inserting said vessel wall cutting tool through said slit opening and adjacent to said first end opening;
e) attaching said first end opening to said blood vessel;
f) excising a piece of blood vessel wall with said vessel wall cutting tool;
g) removing said excised piece of vessel wall and said vessel wall cutting tool from said first end opening and from said slit opening; and
h) closing said slit opening in said conduit.

11. The method of claim 10, wherein said slit opening is defined by a pair of opposing edges.

12. The method of claim 11, wherein said slit opening is closed with a closing means.

13. The method of claim 12, wherein said closing means comprises a suture.

14. The method of claim 13, wherein said pair of opposing edges are hemmed edges, and wherein said suture is sewn through said hemmed edges.

15. The method of claim 14, wherein closing said slit opening in said conduit comprises pulling said suture.

16. The method of claim 12, wherein said closing means comprises one or more staples.

17. The method of claim 12, wherein said closing means comprises one or more clips.

18. The method of claim 12, wherein said slit opening extends through about 5/9ths of a perimeter of said conduit.
